(19) United States
(12) Patent Application Publication
(10) Pub. No.: US 2026/0196310 A1
(43) Pub. Date: Jul. 9, 2026

(54) BLENDED DESCRIPTOR BASED MODELING OF HIGHLY FORMULATED PRODUCTS

(71) Applicants: Dow Global Technologies LLC, Midland, MI (US); Rohm and Haas Company, Collegeville, PA (US)

(72) Inventors: Michael Q. Tran, Wayne, PA (US); Sun Hye Kim, Houston, TX (US); Jonathan P. DeRocher, Coopersburg, PA (US); Kevin J. Henderson, Collegeville, PA (US); Birgit Braun, Pearland, TX (US); Steven G. Arturo, Bala Cynwyd, PA (US); Wenqin Wang, Paoli, PA (US)

(21) Appl. No.: 19/132,904

(22) PCT Filed: Dec. 7, 2022
(86) PCT No.: PCT/US2022/052128

§ 371 Date: May 27, 2025

(51) Int. Cl. G16C 20/30; G16C 20/70

(52) U.S. Cl. CPC G16C 20/30; G16C 20/70

(57) ABSTRACT

Disclosed is a method for blended descriptor based modeling of highly formulated products such as paint. The method includes categorizing the components of the data set into a multi-level classification to produce a reduced data set, incorporating one or more descriptors associated with the components into the reduced data set to generate a modified data set, receiving a prediction of a property of the product from a machine learning module, and adjusting a chemical formulation and/or process generating the product or rejecting the product based on the prediction of the property of the product.

### TECHNICAL FIELD

The present disclosure relates to blended descriptor based modeling of highly formulated products. Such techniques can be particularly useful to predict product properties in order to adjust a chemical formulation used to produce the product or to determine whether to reject a particular chemical formulation to produce the product.

### BACKGROUND

Modern chemical products are often highly formulated to contain large numbers of components having various functions and composition. For example, a conventional architectural paint may contain 10 to 20 individual components, which range from inorganic pigments, binders, functional additives such as dispersants, rheology modifiers, adhesion promoters, and the like. The individual components form a large formulation space in which combinations and specific component quantities are selected to balance desirable properties for each application. Further complexities include variance in the performance of a product between formulations containing differing components of the same type. In the case of the extenders calcium carbonate and silica, substitution between similarly sized components can lead to changes in performance as other features such as sphericality, surface roughness, free ions, and the like promote differing interactions among other components.

In the case of binders, synthesis has as much or even greater effect than the composition of the binder itself. When conceptualizing complex formulations such as architectural paints, the combinatorial vastness (e.g., very high dimensionality) of the space would require large data sets to accurately capture the dynamics of the system. In addition to the vastness of the formulation space, the time and energy required to generate data should not be discounted. Many researchers would consider the time and energy required to generate a sufficient amount of data to properly describe the space untenable.

One approach that is currently being employed is to limit the scope of the formulation space or to limit the complexity of the outputs that are desired. Constraining the formulation space often leads to models that have limited breadth in applicability.

### SUMMARY OF THE DISCLOSURE

The present disclosure is directed to using improvements in machine learning technology to predict a property of a product generated by a chemical process. The prediction can be based on a data set that has been categorized based on material type and that includes a plurality of descriptors associated with the components of the product. The data set including the plurality of descriptors can be generated and input to an artificial neural network (ANN) trained to predict the property of the product based on the components of the product. In addition, the data set including the plurality of descriptors can be generated and input to an ANN trained to predict the components for a product that includes a desired property.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 illustrates one example model 100 to reduce a data set associated with components based on a material type of the components.

FIG. 2 is one example diagram illustrating an approach to produce a reduced data set from an initial data set and add characteristic descriptors of the components to the reduced data set.

FIG. 3 is one example diagram illustrating an approach to add characteristic descriptors of the components into a model that can be utilized as part of a machine learning module.

FIG. 4 illustrates an example of a method for descriptor based modeling.

FIG. 5 illustrates an example of a machine readable medium for descriptor based modeling.

FIG. 6 illustrates an example of a device for descriptor based modeling.

### DETAILED DESCRIPTION

The present disclosure relates to methods and devices for blended descriptor-based modeling of highly formulated products, which may utilize machine learning models to predict product properties for one or more prospective formulations.

A machine learning model can be a function or equation for identifying patterns in data. A machine learning module can be a plurality of machine learning models utilized together to identify patterns in data. In a specific example, a machine learning module can be organized as a neural network. A neural network can include a set of instructions that can be executed to recognize patterns in data. Some neural networks can be used to recognize underlying relationships in a set of data in a manner that mimics the way that a human brain operates. A neural network can adapt to varying or changing inputs such that the neural network can generate a best possible result in the absence of redesigning the output criteria.

A neural network can include multiple neurons, which can be represented by one or more equations or functions. In the context of neural networks, a neuron can receive a quantity of numbers or vectors as inputs and, based on properties of the neural network, produce an output. For example, a neuron can receive Xₖ inputs, with k corresponding to an index of inputs. For each input, the neuron can assign a weight vector, Wₖ, to the input. The weight vectors (e.g., weight value, etc.) can, in some embodiments, make the neurons in a neural network distinct from one or more different neurons in the network. In some neural networks, respective input vectors can be multiplied by respective weight vectors to yield a value, as shown by Equation 1, which shows an example of a linear combination of the input vectors and the weight vectors.

$$f ( {x}_{1} , {x}_{2} ) = {w}_{1} {x}_{1} + {w}_{2} {x}_{2} \qquad Equation 1$$

In some neural networks, a non-linear function (e.g., an activation function) can be applied to the value f(x₁, x₂) that results from Equation 1. An example of a non-linear function that can be applied to the value that results from Equation 1 is a rectified linear unit function (ReLU). Application of the ReLU function, which is shown by Equation 2, yields the value input to the function if the value is greater than zero, or zero if the value input to the function is less than zero. The ReLU function is used here merely as an illustrative example of an activation function and is not intended to be limiting. Other non-limiting examples of activation functions that can be applied in the context of neural networks can include sigmoid functions, binary step functions, linear activation functions, hyperbolic functions, leaky ReLU functions, parametric ReLU functions, softmax functions, and/or swish functions, among others.

$$ReLU ( x ) = max ( x , 0 ) \qquad Equation 2$$

During a process of training a neural network, the input vectors and/or the weight vectors can be altered to “tune” the network. In at least one example, a neural network can be initialized with random weights. Over time, the weights can be adjusted to improve the accuracy of the neural network. This can, over time yield a neural network with high accuracy. The present disclosure utilizes machine learning such as neural networks for predicting product properties through modeling of input data. In these embodiments, the weights can be tuned based on a number of factors. For example, the weights can be tuned for a descriptor based on a quantity of a component added (e.g., weight fraction, etc.) that includes the descriptor.

Embodiments of the present disclosure include blended descriptor based modeling of highly formulated products utilizing machine learning modules such as neural networks. Methods disclosed herein may be used to improve product property predictions from a limited data set for multi-component chemical formulations in varied applications including compositions for paint, home, and personal care. Product property predictions may be generated from one or more machine learning models such as neural networks, random forest, or others, and may include single or multiple qualitative or quantitative values. Methods disclosed herein may also be applied to inverse strategies in which target product properties are input into one or more machine learning modules, which generate an output of one or more prospective product formulations.

Prior to training a machine learning model, formulation components may be organized into a multi-level classification to produce a reduced data set. Multi-level classifications disclosed herein may include two or more classes arranged into one or more levels. At a first level, classes are generated for each component (e.g., binder, surfactant, water, pigments, etc.) of arbitrary broadness. As used herein “class” refers to a category used to define the type of component. A model is used to predict one or more target properties, and a goodness of fit is determined. More detailed models are generated by further subdividing classes (e.g., a class of binder into subclasses: epoxy, polyether, vinyl, acrylic, polyurethane; a class of surfactant into subclasses: anionic, cationic, zwitterionic). The model may be further evolved with more specific classifications, depending on the result of fit for the one or more target properties. The number of levels for each classification needed depends on the property being modeled. For example, additional levels of classifications may improve fit where surfactants can be subdivided based on chemical type (e.g., charge, such as anionic or cationic, molecular weight, functionality, etc.) or function (e.g., impact on rheology, emulsion type, etc.). In other examples, a single level can be utilized for a component. For example, a component such as, but not limited to, water can have a single level. Multiple models are generated and sorted (kept/discarded) based on goodness of fit to property data, and associated uncertainties with each model. In some cases, multiple models may be generated and applied as a module, where the result is a combined product of two or more models (e.g., average, weighted average, etc.).

As used herein, the singular forms “a”, “an”, and “the” include singular and plural referents unless the content clearly dictates otherwise. Furthermore, the word “may” is used throughout this application in a permissive sense (e.g., having the potential to, being able to), not in a mandatory sense (e.g., must). The term “include,” and derivations thereof, mean “including, but not limited to.”

As will be appreciated, elements shown in the various embodiments herein can be added, exchanged, and/or eliminated so as to provide a number of additional embodiments of the present disclosure. In addition, as will be appreciated, the proportion and the relative scale of the elements provided in the figures are intended to illustrate certain embodiments of the present invention and should not be taken in a limiting sense.

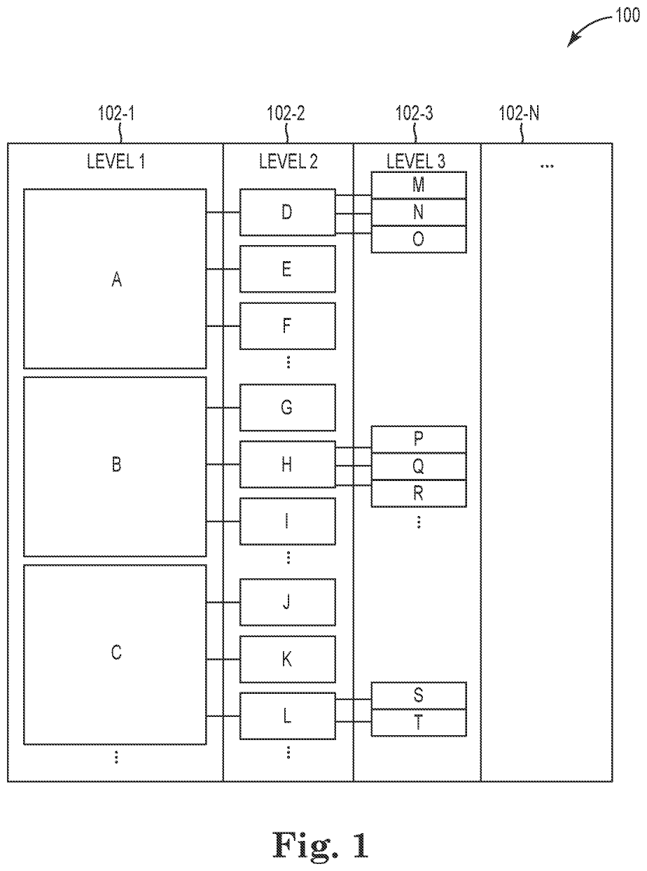

FIG. 1 illustrates one example model 100 to reduce a data set associated with components based on a material type of the components. The lettered boxes in FIG. 1 each represent a different class of components. The classes can be a category or description used to organize components based on a property (e.g., material type, composition, etc.) of the components. The classes are illustrated in different levels 102-1, 102-2, 102-3, . . . , 102-N, which are also referred to as class levels. The model 100 includes a first class level 102-1 (“Level 1”) and may include additional levels, such as second level 102-2 (“Level 2”), third level 102-3 (“Level 3”), and additional levels 102-N. The additional levels under the first level 102-1 may be referred to as subclasses. The subclasses can extend to any number of levels required to capture the relevant component details.

As used herein, a “component” refers to physical matter (e.g., chemical species, reactants, raw materials, etc.) used in the formulation to make the product. The components of the formulation used to make the product (e.g., “end product,” “final product,” etc.) can be described with a plurality of different data metrics in a data set. The data set can include a plurality of different components for generating the product. As described herein, the “data set” can be an exhaustive list of possible components that can be difficult to organize and/or utilize to train a machine learning module.

As described herein, a machine learning module can include a plurality of functions or equations that can be organized as nodes that recognize the underlying relationships in a data set in a manner that mimics the way that a human brain operates. The machine learning module can be trained utilizing the data organized by the model 100. The model 100 can be organized in different levels 102-1, 102-2, 102-3, . . . , 102-N and descriptors can be added to one or more of the classes at the different levels 102-1, 102-2, 102-3, . . . , 102-N. In at least one example, relevant historical formulation data is collected, including names of all components. Components are then categorized using a multi-level class definition of the model 100. In some examples, the descriptors can include at least one of continuous descriptors, ordinal descriptors, binary descriptors, or categorical descriptors. For example, the descriptors can be continuous values, ordinal values, binary values, and/or a categorical value.

The class definition for a component can be used to define the type of component, reactants used to generate the component, among other categorizations of components. A class definition must include a Level 1 class and may include one class for each level below Level 1 within the same branch of the depicted tree structure. Referring to the model 100, some examples of class definitions include (A), (B), (C), (A, D), (A, D, N), and the like. In some examples, each class definition can include one or more descriptors.

As used herein, “descriptor” refers to a quantity or quality attributed to a component in a given class. In one example, a component with a class definition of (A) can have particular properties or attributes that are assigned to the class definition of (A). In this way, the descriptors can be utilized to further describe the qualities or attributes of components designated to a particular class definition. The descriptors can include, but are not limited to: measured characteristics, empirical characteristics, calculated, categorical, and/or compositional characteristics. The measured characteristics can be a measurement that was collected during a particular test of the component. For example, a descriptor “acidic” can be assigned to a particular class. In this example, the components designated to that class will have measured value of a pH that is below 7 under particular conditions. The empirical characteristics can be experimental results of a particular class. The empirical characteristics can be collected for the components through experimental data. The empirical characteristics can be results of combining different quantities of a component or class of components with a different component. In this way, the results of experimentation for a particular class or specific component can be designated to the class or specific component within the model 100. The compositional characteristics can be a compositional property for a class of components. For example, a particular property can be associated with a binder that is an acrylic binder. In this example, the particular property is a compositional property and can be designated to the class of acrylic binder.

In some examples, the descriptors can be calculated descriptors. For example, a calculated descriptor for a pigment of a paint can be calculated based on the quantity and density value of the pigment utilizing the concentration or volume solid of the particular pigment. In these examples, the calculated descriptors can be utilized for a particular component such that the calculation can be applied to the component. In some examples, the descriptors can be categorical descriptors. For example, the descriptor can be a type of monomer (e.g., PEM, MMA, AA, etc.) and whether the monomer is composite forming or not composite forming (e.g., Yes/No category, etc.). In these examples, the categorical descriptors can be one-hot coded based on a yes/no category description and utilize a weighted average when there is a blend or mixture of components (e.g., mixture of different monomers, etc.).

Considering FIG. 1, an example is presented in which a component may have the multi-level class definition of (A), (A, D), or (A, D, N) and so on. The second level 102-2 (e.g., Level 2) classes (D), (E), and (F) are available to the first level 102-1 class of (A); however, as shown in the FIG. 1, the second level 102-2 class (D) can also be available to the first level 102-1 class (B).

Each class at each level may be associated with one or more descriptors. Descriptors may or may not be unique to each class. That is, a particular descriptor may be designated to a plurality of different classes. Each machine learning module may include one or more machine learning models such as artificial neural networks (ANN) such as deep neural networks (DNNs), symbolic regression, recurrent neural networks (RNNs) that include long short-term memory (LSTM) networks or Gated Recurrent Unit (GRU) networks, decision trees, random forests, boosted trees such as gradient boosted trees (XGBoost), linear regression, partial least squares regression, support vector machines, ridge regression, multilayer perceptron (MLP), autoencoders (e.g., denoising autoencoders such as stacked denoising autoencoders), Bayesian networks, support vector machines (SVMs), hidden Markov models (IMNIs), and the like. Commercially available software packages may include JMP software, Microsoft AzureML, SAP data analysis tools, Python, R Project, soft independent modeling by class analogy (SIMCA) by Sartorius, and the like. Although specific types of machine learning models are described, other types of machine learning models could implement descriptors in a similar way.

The methods described further herein may include training or building one or more machine learning modules, and then evaluating the effectiveness of the machine learning modules using a measure of goodness of fit (e.g., R-squared values). Machine learning modules are constructed with a user-defined number of classes and level depth associated with the model. For each module, with reference to FIG. 1, classes may be considered at varying level depths. For example, a first module may be constructed considering components of each class at the first level 102-1 ((A), (B), and (C)). A second module may be constructed in which components in classes (A) and (C) are considered at the first level 102-1, while (B) is considered at the second level 102-2; particularly, the class definitions of (A), (B), (B, G), (B, H), (B, D), and (C). In the second module example, components that do not have the second level 102-2 class definitions of (B, G), (B, H), or (B, D) will retain the first level 102-1 class definition of (B).

In a third module, components in class (A) will be considered at the first level 102-1, components in class (B) will be considered at the second level 102-2, and components in class (C) will be considered at the third level 102-3. In this example, all components will have one of the following class definitions: (A), (B), (B, G), (B, H), (B, D), (C), (C, J), (C, K), (C, L), (C, L, S) and (C, L, T). In this way, a component considered at the first level 102-1 will be defined by the component's categorization at the first level 102-1. In addition, a component considered at the second level 102-2 will be defined by the component's categorization at the first level 102-1, at the second level 102-2, and/or a combination thereof. Furthermore, a component considered at the third level 102-3 will be defined by the component's categorization at the first level 102-1, at the second level 102-2, at the third level 102-3, and/or a combination thereof.

Machine learning modules are constructed/trained using one or more of: component classes, descriptors associated with the class definition of each component, and the component amounts (e.g., quantity of the components, ratios of the components, etc.). As described further herein, depending on the modeling tasks, descriptors may be weighted by the quantity of the component within the product using a range of algebraic approaches (e.g., a generalized mean approach) or used without further modification. The generated machine learning modules are then evaluated singly or as an ensemble (combined results from multiple models) for effectiveness by considering a metric related to the experimental data and evaluating the goodness of fit (e.g., fit value, etc.).

Previous approaches to modeling formulation space typically weight each component as a unique factor. Methods disclosed herein construct machine learning modules that allow for the distillation of specific information or data into the data set as descriptors that allows a subset of the unique components to be generalized into multi-level classes. Generalization reduces the overall data requirement for effective modeling, increasing computational speed while maintaining chemical information and inter-component relationships. For example, instead of including descriptors in the machine learning module for each possible component of a formulation, the number of input variables from the data set is reduced by including descriptors for different generalized classifications of components at different levels of generalization per the model 100. As described herein, the descriptors can be a combination of measured values, chemical properties, empirical chemical relationships, application-specific qualities, and the like.

In a specific example, the descriptors may be used to describe the relationship between different rheology modifiers in a paint formulation. Paints are typically formulated using three characteristic viscosity measurements: cone and plate (ICI), Stormer (KU), and Brookfield. These measurements represent fluid behavior at high shear, medium shear, and low shear, respectively. Depending on the application, the viscosity of the paint formulation is tuned with various rheology modifiers that control viscosity within a particular shear regime or across multiple shear regimes. Rheology modifier efficiency is dependent on a number of factors including chemical structure, polarity, number of active groups, associative behavior (e.g., interaction with other components in the formulation), and rheology modification mechanism.

These factors can be used as descriptors that are used to place the rheology modifiers (components) into a multi-level classification. Descriptors can also include additional behavioral aspects that provide a measure of convoluted formulation behaviors not included by characterization of isolated components. The measure of convoluted formulation behaviors can be a measure of a particular property of a component that corresponds to a particular formulation of the component. That is, a particular component can have a first behavior or first measured behavior when formed in a first methodology while the particular component can have a second behavior or second measured behavior when formed in a second methodology. For example, a rheology modifiers behavior may be included in a class having a descriptor defining the performance of the rheology modifier as a function of its behavior in a reference shear regime such as “ICI modification efficiency at a given Stormer/KU.”

In another example, descriptors may be used to represent empirical relationships and characteristics for binders in paint formulations. Simply describing a binder as an all acrylic or even defining it by its composition does not accurately reflect its behavior/performance. The synthesis of an architectural binder is as much of an art as it is a science due to the kinetics that are used during synthesis as well as the dynamic phenomenon that occur as the result of synthetic procedures. An example of the role kinetics plays in binders can been seen in particle morphology where certain conditions can either cause a homogeneous (one phase) or heterogeneous (multiphasic) morphology. An example of the dynamics in binders can be seen in the expression of polar groups at the surface of the binder particles. In another example, acids can either bury or express themselves as a function of pH or other local interactions at the surface. This phenomenon makes the measurement/quantification of polarity subjective to how the measurement is taken. However, a rough empirical assessment can aggregate the propensity of this phenomenon as it relates to other binders and thus provide information which could not be included into the initial data set.

The use of empirical, measured, calculated, and categorical descriptors provides breadth and depth of information into the data set which maximizes the utility of an otherwise sparse data set. This is particularly the case when each component is used as a column within the data set. Not only is a wealth of domain knowledge excluded from the data set, but this approach drastically increases the dimensionality of the system to be modeled.

The methods described herein overcome the challenges associated with sparse and high-dimensional data sets. Complex formulations often result in small numbers of mixtures with many components, leading to minimal data. Conventional modeling approaches capture component quantities in multiple columns with many columns populated with null values. Many coating components are unique, requiring large numbers for formulations to differentiate performance impact for each component and correspondingly increasing amounts of computing power.

The present disclosure focuses on reducing the complexity of training and testing data sets by generating one or more classes for each component of the formulation. Data dimensions are reduced by replacing component names with class designators and grouping as “Levels”, with the first level 102-1 being the broadest class (e.g., binder, pigment, thickener, etc.), the second level 102-2 including subgroupings by a feature such as component type (e.g., polymer type, function, moiety content, etc.), and continuing to an Nth level 102-N as necessary to characterize the input data. In this way, the reduced data set plus the descriptors can be utilized to generate more accurate machine learning modules for predicting properties of highly formulated products.

That is, the models (e.g., model 100) generated utilizing the multi-level class definitions and characteristic descriptors can improve data utilized by a machine learning module. The machine learning model or machine learning modules utilizing the multi-level class definitions and characteristic descriptors can improve property prediction of highly formulated products compared to utilizing traditional models. Furthermore, the multi-level class definition models described herein provide improved formulation results with selected properties compared to traditional models by condensing the data to reduce complexity and adding descriptors to improve accuracy. In this way, the accuracy of property prediction and/or formulation prediction is improved while also reducing the quantity of computing resources needed to execute the machine learning module.

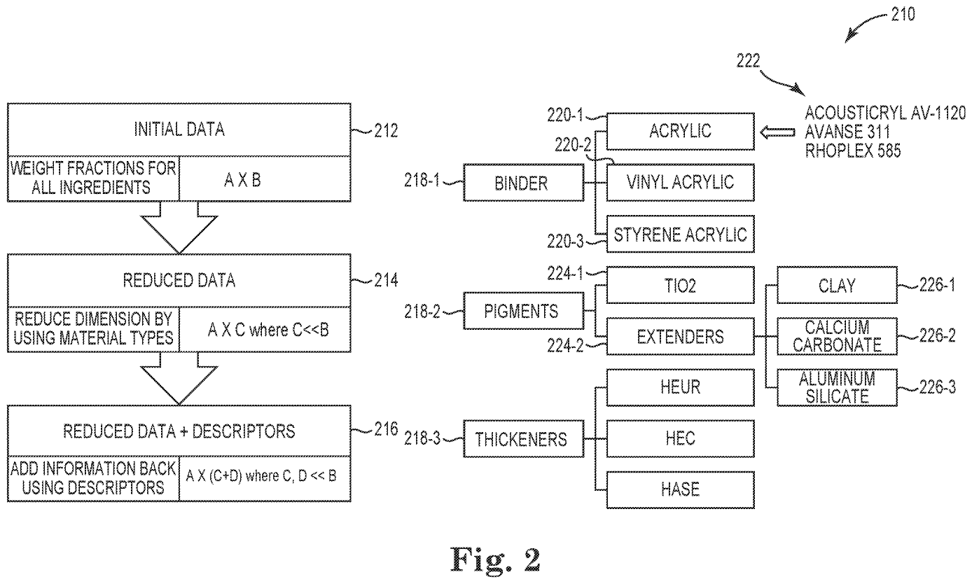

FIG. 2 is one example diagram 210 illustrating an approach to produce a reduced data set 214 from an initial data set 212 and add characteristic descriptors of the components to the reduced data set 214. The diagram 210 includes a similar model to the model 100 illustrated in FIG. 1 along with a method flow from an initial data set 212 to a reduced data set plus descriptors 216. The diagram 210 can illustrate how an initial data set 212 can be reduced to a reduced data set 214. The reduced data set 214 can be categorized into a plurality of levels as illustrated by model 100 as referenced in FIG. 1. As described further herein, the reduced data set 214 can have descriptors added to the plurality of classes and/or levels of the model to generate a reduced data set plus descriptors 216.

In some examples, the initial data set 212 can include data associated with components for a particular product (e.g., compound, mixture, formulation, etc.). For example, the initial data set 212 can include a plurality of components that can be utilized to form a particular end product. In a specific example, the particular product is a highly formulated product such paint. In this example, the paint can include a plurality of components such as, but not limited to: water, a surfactant, a neutralizer, a solvent, a defoamer, a thickener, a binder, a biocide, an extender, a pigment, and/or other components based on the type of paint to be produced. In some examples, a plurality of options can be provided for each of the plurality of components. For example, a plurality of binders can be provided as options for a particular type of binder with corresponding physical and/or chemical properties. In this example, each of the plurality of binder options can have a particular manufacturer with corresponding ratios of reactants to generate the binder. In this way, each of the plurality of components can have a corresponding plurality of options to be utilized to generate the end product (e.g., paint).

The initial data set 212 can include each of the plurality of component options for each of the plurality of components. That is, the initial data set 212 can include all possible component options for each of the plurality of components with corresponding weight fractions. Furthermore, the initial data set 212 can include additional information to describe each of the plurality of options. In this way, the initial data set 212 can be an extensive data set that can include information that may be useful for generating a particular end product as well as information that may not be useful or pertinent for generating the particular end product. In a specific example, the initial data set 212 can include the weight fractions for all of the potential components to generate a particular end product. As used herein, a weight fraction can be represented as a weight of the component over a total weight of the end product.

In some examples, the initial data set 212 can be reduced to the reduced data set 214 by categorizing the components of the initial data set 212 based on material types. That is, each of the plurality of components can be categorized into a particular material type or Level 1 category (e.g., binder 218-1, pigments 218-2, and/or thickeners 218-3 as illustrated by the model) and, for some components. In addition, the plurality of components can be categorized into subclasses of material type or Level 2 categories. For example, the Level 1 category of binder 218-1 can be further categorized into the Level 2 categories of acrylic 220-1, vinyl acrylic 220-2, styrene acrylic 220-3, and the like. The reduction can continue to an N-th Level of subclasses based on the Level 1 class. This reduction can significantly reduce the quantity of information provided by the reduced data set 214 versus the initial data set 212. Descriptors can be added to the reduced data set 214 to increase a quantity and/or quality of information available for each of the plurality of components within each of the Level 1 categories. As used herein, a descriptor is a description of a particular property of a corresponding component that can be designated to a particular level (e.g., Level 1, Level 2, Level N, etc.). In some examples, the descriptors can be based on different combinations of reactants to generate a particular component. As described herein, the different ratios of reactants and/or different combinations of reactants can generate components with different properties that can affect the properties of the end product.

As illustrated in the model, the plurality of components can be categorized into generic categories (e.g., high level description categories, etc.). For example, the model can include a Level 1 category that includes, but is not limited to: a binder 218-1, a pigment 218-2, and a thickener 218-3. Although three of the components are illustrated in the model, additional or fewer components can be utilized to generate or train the machine learning module. A plurality of sub-categories or Level 2 categories can be generated for each of the Level 1 categories. The Level 2 categories can be categories within the Level 1 category. For example, the Level 1 category of a binder 218-1 can include Level 2 categories such as acrylic 220-1, vinyl acrylic 220-2 and/or styrene acrylic 220-3. Although three Level 2 categories are illustrated for the Level 1 category of binder 218-1, additional or fewer Level 2 categories can be utilized. Furthermore, each Level 2 category can include additional Level 3 categories and/or specific components such as components 222. The components 222 can be specific components that can be utilized to generate the end product. Specifically, the components 222 list Acousticryl AV-1120, Avanse 311, and Rhoplex 585, however embodiments herein are not so limited and additional or fewer components 222 could be provided.

In a different example, the Level 1 category can be a pigment 218-2. The pigment 218-2 can include Level 2 categories of titanium dioxide 224-1 and extenders 224-2. Furthermore, the extenders 224-2 Level 2 category can include Level 3 categories that include clay 226-1, calcium carbonate 226-2, and/or aluminum silicate 226-3. In some examples, each of the Level 3 categories or a portion of the Level 3 categories can include Level 4 categories. In contrast, each of the Level 3 categories can be designated with a corresponding list of components.

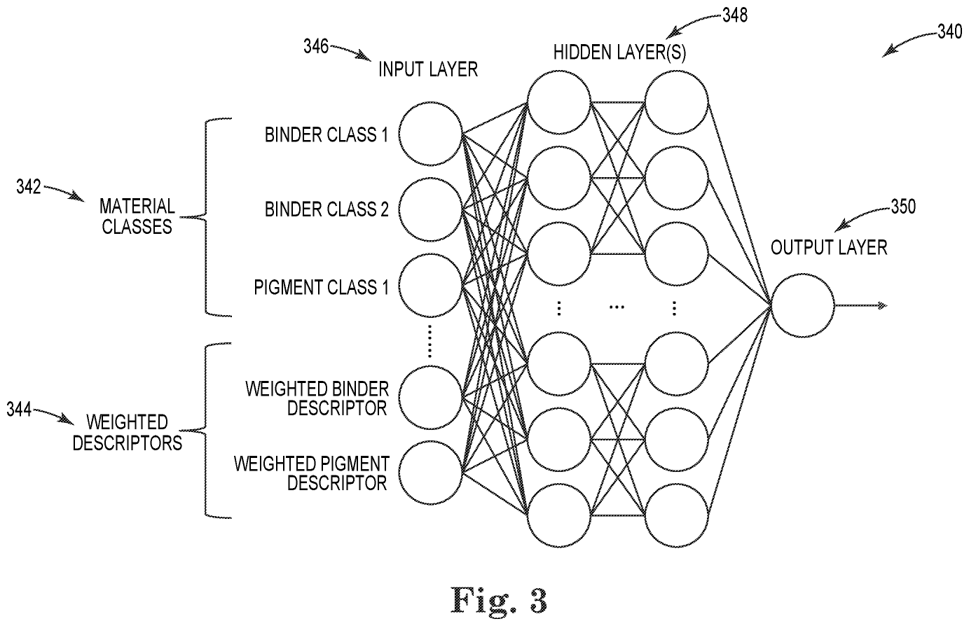

A descriptor or plurality of descriptors can be added to the plurality of categories within each Level. The descriptors can further describe or include information related to the properties of the components within the corresponding category and/or how the components within a corresponding category affect a property of an end product when utilized. In some examples, as will be described further in reference to FIG. 3, the descriptors can add information for different combinations of reactants that are used to generate the corresponding components. As used herein, a combination of a component can include a ratio of reactants and/or a process of formation associated with the component. The combination can include a corresponding list of component properties as described further herein.

As described herein, each component can have different ratios for each reactant combination used to generate the particular component. For example, a binder can have different properties for different combinations of reactants for a particular property such as surface stabilization. A diagram can be generated to illustrate how a particular property for a particular component is divided based on the combination of reactants used to form the component. This type of diagram can be generated for each of the plurality of components. In this way, a first category can illustrate properties of a first binder that is a single binder with a particular surface stabilization. In addition, the diagram can include a second category that can illustrate properties for a second binder that includes a binder mixture with a particular mixture ratio. The properties of the first binder and the second binder can be utilized to predict how the binder will alter or affect a property of the end product.

FIG. 3 is one example diagram 340 illustrating an approach to add characteristic descriptors of the components into a model for a machine learning module. In some examples, the diagram 340 can illustrate an example portion of an ANN or similar machine learning module. In some examples, the diagram 340 includes a plurality of nodes associated with an input layer 346, a plurality of nodes associated with an output layer 350, and a plurality of nodes associated with a hidden layer 348 between the input layer 346 and the output layer 350.

The plurality of nodes can be represented by circles within the hidden layer 348 of the diagram 340. As described herein, the plurality of nodes can act as neurons, which can be represented by one or more equations or functions. In this way, an output value of a particular node of the plurality of nodes can be utilized as an input value for a subsequent node of the plurality of nodes based on the equation or function at the particular node. In another example, the output value of a particular node can be utilized to determine a subsequent node to activate or utilize within the hidden layer 348. In this way, a particular node of the plurality of nodes within the hidden layer 348 can correspond to a particular weight or bias that is to be performed on an input or input value. In some examples, the input layer 346 can include a first plurality of input values that represent material classes 342 and a second plurality of input values that represent weighted descriptors 344. The material classes 342 can be the identified classes of components as described herein and the weighted descriptors 344 can be the descriptors associated with components with corresponding weighted values. In some examples, the input values from the material classes 342 and/or the input values from the weighted descriptors 344 can be provided to the plurality of nodes within the hidden layer 348 or a portion of the plurality of nodes within the hidden layer 348 as input values to generate output values of the output layer 350.

As described herein, the output layer 350 can include values that can be utilized to generate a formulation or plurality of formulations that include a desired property. In some examples, the output layer 350 can include a plurality of different output values. For example, the plurality of output values can correspond to different levels of fit or fit values that can be within an acceptable range for the input values. In some examples, the output values of the output layer 350 can be analyzed to determine a best fit value for the desired property. In some examples, the output values of the output layer 350 can be utilized to modify the plurality of input values of the input layer 346. For example, the output values of the output layer 350 can be utilized to select or deselect particular material classes 342 and/or alter weighted values of the weighted descriptors 344. In this way, the ANN illustrated by the diagram 340 can be further tuned to increase an accuracy of the output values of the output layer 350 generated by the ANN.

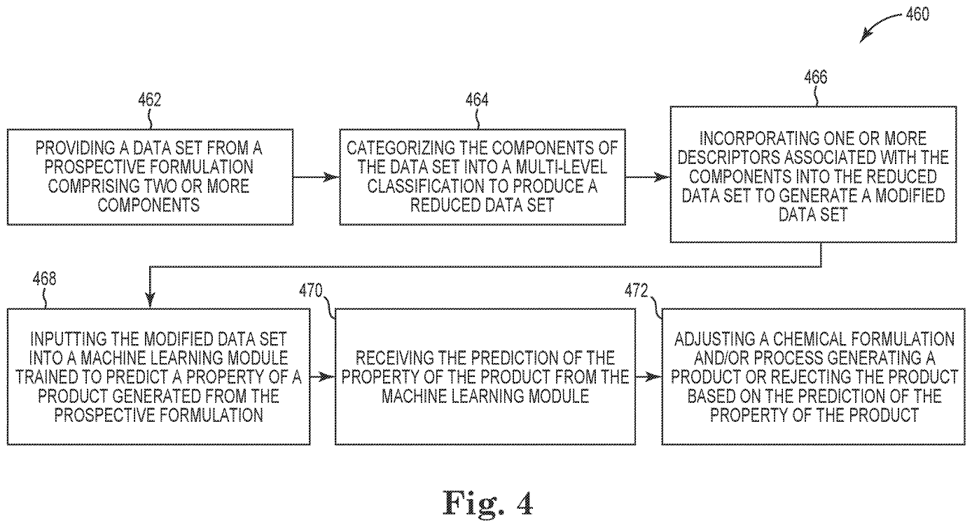

FIG. 4 illustrates an example of a method 460 for descriptor based modeling. The method 460 can be implemented to train one or more machine learning models of a machine learning module or ANN to predict a property of a product comprising a plurality of components. In some examples, the method 460 can be executed by a computing device as described herein. The method 460 can be utilized to adjust chemical formulations and/or adjust the process for generating a desired product. The method 460 can allow a user to optimize or increase the presence of a desired property within a complex or highly formulated product.

At step 462, the method 460 can include providing a data set from a prospective formulation comprising two or more components. As described herein, a data set (e.g., initial data set 212 as referenced in FIG. 2, etc.) can include formulation data, chemical data, physical data, and/or data related to properties of a plurality of components. In this way, the components or formulation of a product can be identified based on the two or more components of the product to be formed. In some examples, the formulation can be utilized to identify the two or more components of the product. In these examples, the data set can include information related to the two or more components.

As described herein, the data set for the two or more components can include weight fractions for all of the components for a particular product generated by the formulation. In this way, the quantity of data within the data set can be relatively large for a highly formulated product. In some examples, the method 460 includes providing a historical data set from historical formulation data. The historical data set from the historical formulation data can be data that has been collected by producing products from the historical formulation data and performing property test on the products. In this way, historical formulations and corresponding product properties can be utilized to determine components that affect a particular property of the product. For example, a portion of the two or more components within the data set may have a relatively greater effect on an end product's sheen property compared to other components.

At step 464, the method 460 can include categorizing the components of the data set into a multi-level classification to produce a reduced data set. As described herein, the multi-level classification can include one or more classes arranged into two or more levels. As illustrated in FIG. 1 and FIG. 2, the multi-level classification can reduce the initial data set by creating different categories at each of the plurality of levels and assigning the plurality of components into one or more of the plurality of levels. For example, the data set can be reduced to a reduced data set (e.g., reduced data set 214, etc.) by categorizing the plurality of components based on material types. In this way, the data set can be more easily managed and implemented into a machine learning module.

In examples that utilize the historical data, the method 460 includes categorizing the components of the historical data set into the multi-level classification to produce the reduced data set. As described herein, the historical data can be data collected through previous experimentation with the plurality of components and/or experiments on resulting products that were generated by the plurality of components. Implementing the historical data set into the multi-level classification can increase a fit value for predicting a property of a product generated by a different formulation.

In some examples, categorizing the data into the one or more classes further includes determining a feature selection for the one or more classes based on their impact on the predicted property. In some examples, the feature selection can be based on values that indicate greater or lesser importance. In this case, the feature selection can be assigned to classes based on the impact or importance associated with the predicted property for components within the one or more classes. In this way, the components within the classes that have a greater value of feature importance can have a higher significance within the categorization compared to components within the classes that have a lower feature importance (e.g., feature importance value, etc.). In some examples, the feature selection can allow some categories to be disregarded or have relatively less impact on the machine learning prediction module than other categories. In some examples, the feature selection allows for material classes to be added or dropped from the machine learning prediction module. For example, a desired property can be a particular level of gloss for a paint. In this example, the gloss of the paint can be highly correlated to a pigment volume concentration (PVC) of the paint. In this way, other material classes, such as rheology modifiers can have less impact on the gloss of the paint and may be dropped from the machine learning prediction module while the pigment volume can be added to the machine learning prediction module.

In some examples, the material classes can be added or dropped from the machine learning prediction module in response to a predicted importance of the material class to a desired property. In other examples, the material classes can be added or dropped from the machine learning prediction module in response to results associated with a plurality of different multi-level classification model utilized to train the machine learning prediction module. In this way, the feature selection for the material classes can be assigned based on a plurality of different factors including the results of a previously executed machine learning prediction module.

In other examples, categorizing the data into one or more classes further includes assigning feature importance values to the one or more classes based on their impact on the prospective formulation. In a similar way to the feature importance assigned based on the impact of the predicted property, the one or more classes can be assigned a feature importance based on the impact on the prospective formulation. In some examples, a particular category of components may not affect the prospective formulation. In these examples, the particular category can be assigned a relatively low feature importance such that computing resources to execute the machine learning prediction module can be relatively lower. In another example, the classes or categories that may have a greater impact on the prospective formulation can be assigned a relatively greater feature importance to ensure the machine learning prediction module is applying a priority to the classes with the greater impact on the prospective formulation.

At step 466, the method 460 can include incorporating one or more descriptors associated with the components into the reduced data set to generate a modified data set. In some examples, the one or more descriptors includes, but is not limited to: measured characteristics, empirical characteristics, or compositional characteristics to produce the modified data set. The one or more descriptors can include data that describes the properties of the components. The descriptors can be assigned or incorporated into the classes of the modified data set. In this way, the descriptors can describe characteristics of the components that are assigned within a particular category. The one or more descriptors can be incorporated into a particular component within the reduced data set if the descriptor is specific to the particular component. In other examples, the descriptors can identify a plurality of combinations for each of a plurality of reactants or reactant ratios to generate the components. In this way, components that have different properties under different conditions can be identified by the descriptors within the reduced data set.

Categorizing the components of the data set can further include generating the one or more descriptors to include property transformation data based on a combination of reactants associated with the components. In these examples, the property transformation data includes a property description for the component based on a ratio of the reactants used to form the component. As described herein, the components used to form the product can have different properties based on how the components were formed. That is, the reactants to generate the components can affect the properties of the components and have a different effect on the properties of the product. For example, the pH of a component can affect particular properties of a product generated by the component. In this example, the formation of the component can affect the pH. In this way, the property transformation data can include descriptions of the different reactants to form the components and the resultant properties of the components for the different reactants or different ratios of reactants.

At step 468, the method 460 can include inputting the modified data set into a machine learning module trained to predict a property of a product generated from the prospective formulation. As described herein, the modified data set with the descriptors can be provided to a machine learning module to be executed by a computing device. In some examples, the machine learning module can identify a fit value for a plurality of different functions and a particular function can be selected based on a complexity value and fit value as described herein. The machine learning module can be trained through values associated with the modified data set. In some examples, a portion of the levels, classes, and/or descriptors can be selected based on the feature importance or weighted averages to generate a function with a fit value above a threshold fit value.

In some examples, the method 460 includes inputting the modified data set of the one or more classes into the machine learning module to produce an updated trained machine learning module to predict the property of the product. As described herein, the historical data can be added to the modified data set to be provided to the machine learning module. The machine learning module can be trained utilizing the historical data added to the modified data set to produce the updated trained machine learning module. In some examples, the updated trained machine learning module can generate relatively higher fit values compared to the previous machine learning module.

At step 470, the method 460 can include receiving the prediction of the property of the product from the machine learning module. In these examples, receiving the prediction of the property of the product can further include receiving the prediction of one of a group of properties including molecular weight, density, quality, performance, and identification. In some examples, the prediction of the property can be a prediction for a selected property. For example, a surface stabilization category can be selected and the prediction of the surface stabilization category for a product utilizing the prospective formulation can be calculated. In this way, a desired property can be selected and the machine learning module can produce a value associated with the desired property based on the modified data set and/or the modified data set that includes the historical data set.

In some cases, the method 460 can move from step 470 to step 464 with a different multilevel classification. In these examples, the method 460 can execute steps 466, 468, 470 utilizing the different multilevel classification. In these examples, the method 460 can include a step to compare the results of the first multilevel classification to the different multilevel classification. In some examples, the method 460 performs steps 466, 468, and 470 a plurality of additional times utilizing a plurality of different multilevel classifications to obtain a plurality of results that can be compared.

In some examples, the method 460 includes steps to compare the plurality of results from the plurality of different multilevel classifications to determine a best fit model for a particular desired property. Based on the comparison, the method 460 can move to step 472.

At step 472, the method 460 can include adjusting a chemical formulation and/or process generating a product or rejecting the product based on the prediction of the property of the product. In some examples, adjusting the chemical formulation includes receiving output suggestions from the machine learning module to alter the weight fractions for particular components. For example, the machine learning module can generate a value for a predicted property. In this example, the machine learning module can generate output suggestions for components to alter, add, or remove to alter the predicted property to a desired property. In this way, the chemical formulation can be adjusted to improve a predicted property value for a product to be produced.

In another example, the process for generating the product can also be adjusted. In some examples, the machine learning module can generate output suggestions to alter, add, or remove particular steps of a process for generating the product that could improve the desired property of the product. In some examples, the specifications of the process can be altered based on suggestions provided by the machine learning module. For example, a temperature of a particular process can be altered based on a suggestion from the machine learning module. The steps of the process can be analyzed from the historical data to determine how particular steps of the process affect a particular property of the product. In this way, the process can be altered to improve the desired property of the product.

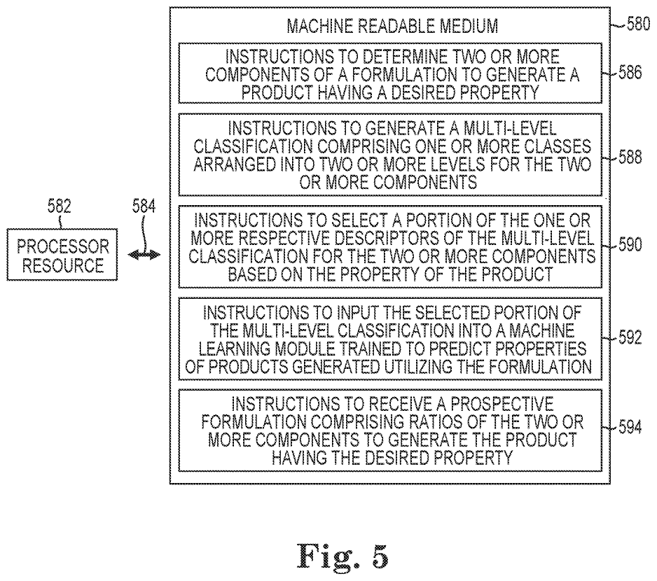

FIG. 5 illustrates an example of a machine readable medium 580 for descriptor based modeling. The machine readable medium 570 can be communicatively connected to a processor resource 582 by a communication path 584. In some examples, a communication path 584 can include a wired or wireless connection that can allow communication between devices and/or components within a single device. As used herein, the processor resource 582 can include, but is not limited to: a central processing unit (CPU), an application specific integrated circuit (ASIC), a field programmable gate array (FPGA), a metal-programmable cell array (MPCA), a semiconductor-based microprocessor, or other combination of circuitry and/or logic to orchestrate execution of instructions 586, 588, 590, 592, 594. In a specific example, the processor resource 582 utilizes a non-transitory computer-readable medium storing instructions 586, 588, 590, 592, 594, that, when executed, cause the processor resource 582 to perform corresponding functions.

The machine readable medium 580 may be electronic, magnetic, optical, or other physical storage device that stores executable instructions. Thus, a non-transitory machine-readable medium (MRM) (e.g., machine readable medium 580) may be, for example, a non-transitory MRM comprising Random-Access Memory (RAM), read-only memory (ROM), an Electrically Erasable Programmable ROM (EEPROM), a storage drive, an optical disc, and the like. The machine readable medium 580 may be disposed within a controller and/or computing device. In this example, the executable instructions 586, 588, 590, 592, 594, can be “installed” on the device. Additionally, and/or alternatively, the machine readable medium 580 can be a portable, external, or remote storage medium, for example, which allows a computing system to download the instructions 586, 588, 590, 592, 594, from the portable/external/remote storage medium. In this situation, the executable instructions may be part of an “installation package”.

The machine readable medium 580 includes instructions 586 to determine two or more components of a formulation to generate a product having a desired property. In some examples, a particular product with a desired property can be selected. The particular product can include a general formulation or have a set of components that are normally utilized to generate the particular product. The desired property can be a property of the particular product that can be affected or altered based on the formulation. For example, a property such as drying time for a paint product may be altered based on the components utilized to generate the paint product. In this example, a generic paint product can have a plurality of components that can be utilized. However, the weight fractions and/or ratios of the plurality of components can be altered to alter particular properties. In this way, the selection of the product can be utilized to generate the components while the selection of the desired property can be utilized to identify the components that alter the desired property.

The machine readable medium 580 includes instructions 588 to generate a multi-level classification comprising one or more classes arranged into one or more levels for the two or more components. In some examples, the multi-level classification includes one or more respective descriptors for each of the two or more components. In these examples, the one or more descriptors include one or more characteristics of products associated with corresponding combinations of reactants to generate the two or more components. In some examples, the one or more descriptors describe a corresponding characteristic of a product associated with different ratios of the combination of reactants. As described herein, the multi-level classification can be a model of a plurality of possible components that can be utilized to generate the product.

The machine readable medium 580 can include instructions to generate a weighted value for the two or more components based on an effect of altering the identified property. In these examples, a greater weight value is assigned to a component with a relatively greater effect of altering the identified property and a lower weight value is assigned to a component with a relatively lower effect on altering the identified property. As described herein, the weighted value can be assigned to a component and/or a descriptor of components based on an affect associated with the component or class of components with the desired property. In order to alter a property of the product to have the desired property, the components with a greater effect in altering the property can be provided with a greater weight or priority.

The machine readable medium 580 includes instructions 590 to select a portion of the one or more respective descriptors of the multi-level classification for the two or more components based on the property of the product. In some examples, the descriptors can include a plurality of combinations or cases that identify different properties for the component based on the formulation of the component. In this way, a particular component can have different properties based on how the particular component was formed. In this example, a first combination for a particular component can have a relatively large effect on generating the product with the desired property while a second combination for the particular component can have a relatively low affect or lower effect on generating the product with the desired property. In this example, the first combination descriptor can be selected over the second combination descriptor.

The machine readable medium 580 can include instructions to select the portion of the one or more descriptors of the multi-level classification based on the weighted value assigned to the one or more descriptors. As described herein, different descriptors can have different levels of importance or effect on the generating a product with the desired property. In this way, the descriptors can be assigned a particular weight value and the portion of the descriptors that are selected can be based on the weight value.

The machine readable medium 580 includes instructions 592 to input the selected portion of the multi-level classification into a machine learning module trained to predict properties of products generated utilizing the formulation. As described herein, the selected portion of the multi-level classification can have a relatively greater effect on generating the product with the desired property compared to other portions of the multi-level classification. The selected portion can be provided to the machine learning module to predict properties of products generated under different formulations and/or different processes.

The machine readable medium 580 includes instructions 594 to receive a prospective formulation comprising ratios of the two or more components to generate the product having the desired property. In some examples, the machine learning module can generate weight fractions for the components of the formulation for generating the product with the desired properties. In these examples, a plurality of formulations with property ranges within the desired property range can be provided. For example, the selected portion of the multi-level classification can be associated with a first portion of components and a non-selected portion of the multi-level classification can be associated with a second portion of components. In this example, the weighted fractions of the first portion of components can be relatively more specific than the second portion since the first portion has a greater effect on generating the desired property of the product.

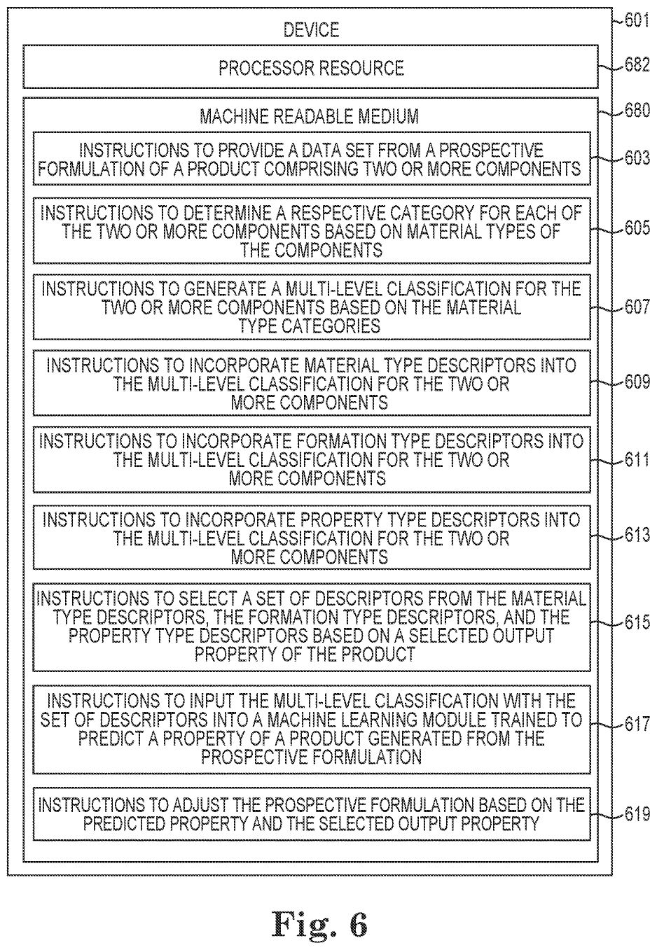

FIG. 6 illustrates an example of a device 601 for descriptor based modeling. In some examples, the device 601 is a computing device that includes a processor resource 682 and a machine readable medium 680 to store instructions 603, 605, 607, 609, 611, 613, 615, 617, 619 that are executed by the processor resource 682 to perform particular functions. FIG. 6 illustrates how a computing device can execute instructions to perform functions described herein.

The device 601 includes instructions 603 stored by the machine readable medium 680 that is executed by the processor resource 682 to provide a data set from a prospective formulation of a product comprising two or more components. The data set from the prospective formulation can be an initial data set (e.g., initial data set 212, etc.). As described herein, the initial data set can be a data set that includes weight fractions for all components of a plurality of different formulations for different end products. As described herein, the initial data set can include historical data from experiments or tests to determine properties of the different products generated by the different formulations. In this way, property values associated with the historical data can be part of the initial data set.

The device 601 includes instructions 605 stored by the machine readable medium 680 that is executed by the processor resource 682 to determine a respective category for each of the two or more components based on material types of the components. As described herein, the data set or initial data set can be reduced by categorizing the components based on material types of the components. In some examples, the categorization of the data set can be utilized to generate a reduced data set (e.g., reduced data set 214 as referenced in FIG. 1, etc.).

The device 601 includes instructions 607 stored by the machine readable medium 680 that is executed by the processor resource 682 to generate a multi-level classification for the two or more components based on the material type categories. In some examples, the multi-level classification of the categories of the reduced data set can be implemented as a model (e.g., model 100 as referenced in FIG. 1, etc.). As described herein, the multiple levels can each be different categories based on the material type of a previous category. In some examples, the plurality of levels can be based on the classifications of a particular material type.

The device 601 can include instructions to generate a plurality of categories that correspond to a plurality of material types for the multi-level classification. In these examples, the device 601 can include instructions to assign a different component to a corresponding category of the plurality of categories based on a material type of the different component. Furthermore, the device 601 can include instructions to incorporate a plurality of descriptors to the multi-level classification for the different component based on a set of properties associated with the different component. In this way, the multi-level categorization or model can be updated to utilize additional components that are not in a previous model.

The multi-level categorization model can include a plurality of classes that are organized into a plurality of levels. The plurality of classes can be designated with one or more descriptors. The descriptors can be utilized to incorporate empirical data, measurement data, and/or compositional data into the model. In some examples, the empirical data can be incorporated into the model through material type descriptors to describe a type of material associated with a component or class of components, a formation type descriptor to describe the formation associated with a component or class of components, and/or property type descriptors to describe a particular property associated with a component or class of components.

The device 601 includes instructions 609 stored by the machine readable medium 680 that is executed by the processor resource 682 to incorporate material type descriptors into the multi-level classification for the two or more components. The material type descriptors can be incorporated to further describe material properties of the components within particular classes or levels. The material type descriptors can include chemical formulations, particle size, molecular mass, density, hardness, pH, melting point, boiling point, and/or other properties of the components. In some examples, the material type descriptors can be limited to features or properties that affect a particular product or type of product to be formed.

The device 601 includes instructions 611 stored by the machine readable medium 680 that is executed by the processor resource 682 to incorporate formation type descriptors into the multi-level classification for the two or more components. As used herein, the formation type descriptors can be descriptions of properties associated with a component that was formed by different processes and/or different reactants. As described herein, a particular component can have different component properties based on how the particular component was formed. As described herein, the component properties can affect the properties of the end product. In this way, the formation type descriptors can be utilized to describe the component properties of a component under different formation conditions.

The device 601 includes instructions 613 stored by the machine readable medium 680 that is executed by the processor resource 682 to incorporate property type descriptors into the multi-level classification for the two or more components. The property type descriptors can describe how the component affects a particular property of an end product when utilized to form the end product. For example, the property type descriptors can identify or describe how the component will affect a sheen of a paint product. In other examples, the property type descriptors can identify different ratios of the component within the end product and how a particular property is affected by the different ratios of the component within the end product.

The device 601 includes instructions 615 stored by the machine readable medium 680 that is executed by the processor resource 682 to select a set of descriptors from the material type descriptors, the formation type descriptors, and the property type descriptors based on a selected output property of the product. The device 601 can include instructions to rank the material type descriptors, the formation type descriptors, and the property type descriptors based on the selected output property. As described herein, a weighted value or priority level can be assigned to the plurality of descriptors. The weighted value can be utilized to rank the different descriptors. In some examples, the set of descriptors can be assigned the weighted value and/or ranked based on the selected output property or desired property of the product. That is, the selected output property can be a particular desired product value or desired property range for the product and the set of descriptors can be selected based on how the set of descriptors affects the desired property.

In some examples, the set of descriptors are each positioned at a different level of the multi-level classification. In some examples, the set of descriptors can be assigned to different levels of the multi-level classification or model since a first descriptor assigned to Level 1 may be relevant to the selected property of the product and a second descriptor assigned to Level 3 may also be relevant to the selected property.

In these examples, the device 601 can include instructions to incorporate numerical data descriptors for the two or more components based on the set of descriptors. Numerical data descriptors can be numerical property data based on historical data and/or tests that were performed on the components or products generated by the components. For example, the numerical data descriptors can be pH values, hardness values, pigment values, among other numerical values or ranges of values that can be utilized to describe a property or feature of a component.

The device 601 can include instructions to generate a set of ratios for combining the two or more components to generate the product with the predicted property. For example, the set of ratios can be a weight fraction for a formulation to generate the product with the predicted property. In some examples, the weight fraction or set of ratios can be utilized for formulations to generate the components and utilized for formulations to generate the product utilizing the components.

The device 601 can include instructions to incorporate performance type descriptors into the multi-level classification for the two or more components to identify different resultant performances for the different reactant combinations that form the two or more components based on performance data for different products utilizing a component within the same categories as the two or more components. The performance type descriptors can be performance data for a particular component that was generated by a particular combination of reactants and/or particular process. As described herein, the performance type descriptor can be description or category designated based on the performance data. For example, a performance type can be designated for components that fall within a particular range of values for a particular performance test.

The device 601 includes instructions 617 stored by the machine readable medium 680 that is executed by the processor resource 682 to input the multi-level classification with the set of descriptors into a machine learning module trained to predict a property of a product generated from the prospective formulation. As described herein, the multi-level classification and/or model can be utilized to train the machine learning module to improve the accuracy of the machine learning module in predicting the property of the product. In these examples, the predicted property can be compared to the selected output property.

The device 601 includes instructions 619 stored by the machine readable medium 680 that is executed by the processor resource 682 to adjust the prospective formulation based on the predicted property and the selected output property. As described herein, the predicted property can be compared to the selected output property and the prospective formulation can be altered if the predicted property is not within a particular threshold of the selected output property. In this way, the prospective formulation can be adjusted to alter the predicted property closer to or within the range of the selected output property.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Various advantages of the present disclosure have been described herein, but embodiments may provide some, all, or none of such advantages, or may provide other advantages.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method, comprising:
providing a data set from a prospective formulation comprising two or more components;
categorizing the components of the data set into a multi-level classification to produce a reduced data set, the multi-level classification comprising one or more classes arranged into one or more levels, wherein at least one class includes two or more levels;
incorporating one or more descriptors associated with the components into the reduced data set to generate a modified data set, wherein the one or more descriptors include composition characteristics of the components;
inputting the modified data set into a machine learning module trained to predict a property of a product generated from the prospective formulation;
receiving the prediction of the property of the product from the machine learning module; and
adjusting a chemical formulation and/or process generating a product or rejecting the product based on the prediction of the property of the product.

2. The method of claim 1, further comprising training the machine learning module by:
providing a historical data set from historical formulation data;
categorizing the components of the historical data set into the multi-level classification to produce the reduced data set;
incorporating the one or more descriptors into the reduced data set, the one or more descriptors comprising at least one of: measured characteristics, empirical characteristics, calculated parameters, or compositional characteristics to produce the modified data set; and
inputting the modified data set of the one or more classes into the machine learning module to produce an updated trained machine learning module to predict the property of the product.

3. The method of claim 1, wherein categorizing the data into the one or more classes further comprises assigning a feature importance to the one or more classes based on their impact on the predicted property.

4. The method of claim 1, wherein receiving the prediction of the property of the product comprises receiving the prediction of one of a group of properties including molecular weight, density, quality, performance, and identification.

5. The method of claim 1, wherein categorizing the data into one or more classes further comprises assigning a feature importance to the one or more classes based on their impact on the prospective formulation.

6. The method of claim 1, wherein categorizing the components of the data set further comprises generating the one or more descriptors to include property transformation data based on a combination of reactants associated with the components.

7. The method of claim 6, wherein the property transformation data includes a property description for the component based on a ratio of the reactants used to form the component.

8. A machine-readable medium, storing machine-readable instructions which, when executed by a processor resource of a device, cause the processor to:
determine two or more components of a formulation to generate a product having a desired property;
generate a multi-level classification comprising one or more classes arranged into one or more levels for the two or more components, wherein the multi-level classification includes one or more respective descriptors for each of the two or more components;
select a portion of the one or more respective descriptors of the multi-level classification for the two or more components based on the property of the product;
input the selected portion of the multi-level classification into a machine learning module trained to predict properties of products generated utilizing the formulation; and
receive a prospective formulation comprising ratios of the two or more components to generate the product having the desired property.

9. The machine-readable medium of claim 8, wherein the one or more descriptors include one or more characteristics of products associated with corresponding combinations of reactants to generate the two or more components.

10. The machine-readable medium of claim 9, wherein the one or more descriptors describe a corresponding characteristic of a product associated with different ratios of the combination of reactants.

11. The machine-readable medium of claim 8, comprising instructions to generate a weighted value for the two or more components based on an effect of altering the desired property, wherein a greater weight value is assigned to a component with a relatively greater effect of altering the identified property and a lower weight value is assigned to a component with a relatively lower effect on altering the identified property.

12. The machine-readable medium of claim 8, wherein the descriptors include at least one of continuous descriptors, ordinal descriptors, binary descriptors, or categorical descriptors.

13. The machine-readable medium of claim 11, comprising instructions to select the portion of the one or more descriptors of the multi-level classification based on the weighted value assigned to the one or more descriptors.

14. A device, comprising:
a processor resource; and
a non-transitory memory resource storing machine-readable instructions stored thereon that, when executed, cause the processor resource to:
provide a data set from a prospective formulation of a product comprising two or more components;
determine a respective category for each of the two or more components based on material types of the components;
generate a multi-level classification for the two or more components based on the material type categories, wherein the multi-level classification comprises one or more classes arranged into one or more levels, wherein at least one class includes two or more levels;
incorporate material type descriptors into the multi-level classification for the two or more components to identify material properties of the two or more components at a corresponding level of the multi-level classification;
incorporate formation type descriptors into the multi-level classification for the two or more components to identify different reactant combinations to form the two or more components;
incorporate property type descriptors into the multi-level classification for the two or more components to identify different resultant properties for the different reactant combinations that form the two or more components;
select a set of descriptors from the material type descriptors, the formation type descriptors, and the property type descriptors based on a selected output property of the product;
input the multi-level classification with the set of descriptors into a machine learning module trained to predict a property of a product generated from the prospective formulation; and
adjust the prospective formulation based on the predicted property and the selected output property.

15. The device of claim 14, wherein the processor is to rank the material type descriptors, the formation type descriptors, and the property type descriptors based on the selected output property.

16. The device of claim 14, wherein the processor is to generate a set of ratios for combining the two or more components to generate the product with the predicted property.

17. The device of claim 14, wherein the processor is to incorporate performance type descriptors into the multi-level classification for the two or more components to identify different resultant performances for different reactant combinations that form the two or more components based on performance data for different products utilizing a component within the same categories as the two or more components.

18. The device of claim 14, wherein the processor is to:
generate a plurality of categories that correspond to a plurality of material types for the multi-level classification;
assign a different component to a corresponding category of the plurality of categories based on a material type of the different component; and
incorporate a plurality of descriptors to the multi-level classification for the different component based on a set of properties associated with the different component.

19. The device of claim 14, wherein the processor is to incorporate numerical data descriptors for the two or more components based on the set of descriptors.

20. The device of claim 14, wherein the set of descriptors are each positioned at a different level of the multi-level classification.
